(19) United States
(12) Patent Application Publication
(10) Pub. No.: US 2018/0079779 A1
(43) Pub. Date: Mar. 22, 2018

(54) METHOD FOR THE PURIFICATION OF A THERAPEUTIC RECOMBINANT PROTEIN FROM TRANSGENIC MILK

(71) Applicant: Laboratoire Francais du Fractionnement et des Biotechnologies, Les Ulis (FR)

(72) Inventor: Sandrine DE MARCO, Paris (FR)

(73) Assignee: Laboratoire Francais du Fractionnement et des Biotechnologies, Les Ulis (FR)

(21) Appl. No.: 15/562,134

(22) PCT Filed: Apr. 1, 2016
(86) PCT No.: PCT/FR2016/050739

§ 371 Date: Sep. 27, 2017

(30) Foreign Application Priority Data
Apr. 2, 2015 (FR) 1552850

(51) Int. Cl. C07K 1/36; C07K 1/14; C07K 1/16; C07K 16/04; C07K 1/34; C07K 16/24

(52) U.S. Cl. CPC C07K 1/36; C07K 1/14; C07K 1/165; C07K 2317/24; C07K 1/34; C07K 16/241; C07K 2317/12; C07K 16/04

(57) ABSTRACT

The invention relates to a method for the purification of a therapeutic protein from transgenic milk, comprising a clarification step using a salt of poly(diallyldimethylammonium).

### FIELD OF THE INVENTION

The invention relates to the purification of therapeutic proteins produced recombinantly in a biological fluid obtained from a transgenic animal.

### TECHNOLOGICAL BACKGROUND

Biopharmaceuticals, in particular therapeutic proteins, are a major source of innovation and represent today nearly half of the new molecules registered worldwide. In May 2013, nearly 170 biopharmaceuticals were marketed in France, among which recombinant monoclonal antibodies constitute an important therapeutic class, in particular for treating cancer and certain autoimmune diseases or for preventing graft rejection (Leem, Biopharmaceuticals in France: the state of the art, 2013 [In French]).

The vast majority of therapeutic proteins are recombinantly produced. Numerous expression systems exist, such as unicellular organisms (bacteria or yeasts), insect cells (baculovirus/insect cell system) or transgenic plants. Nevertheless, these expression systems have many limitations, in particular in connection with imperfect protein folding, the impossibility of producing complex proteins such as immunoglobulins, or glycosylation that is incomplete or different from that found in man.

The expression systems most commonly used in the pharmaceuticals industry are, at the present time, mammalian cells. By way of example, the active ingredient of MabThera®, a chimeric anti-CD20 antibody for treating non-Hodgkin lymphoma, is produced recombinantly in the CHO (Chinese Hamster Ovary) cell line.

Mammalian cells can be used to produce recombinant proteins with glycosylation patterns very similar to those of endogenous human proteins, but generally they provide low production yields.

The industrial need to produce therapeutic proteins with high yield has led the pharmaceuticals industry to consider the use of transgenic mammals, such as cows, rabbits or goats, as expression systems. In this approach, expression of the recombinant protein is directed at mammary epithelial cells. The recombinant protein is thus secreted in the milk and can be recovered from this fluid by extraction and purification processes. By way of example, Atryn®—a recombinant human antithrombin approved by the FDA and the EMEA for prophylactic treatment of venous thromboembolism in patients with congenital antithrombin deficiency—is produced in the milk of transgenic goats (Houdebine, Comparative Immunology Microbiology & Infectious Disease, 2009, 32, 107-121).

Several applications and patents describe the preparation of therapeutic proteins in transgenic milk. Notably, patent EP 0 741 515 and application WO2004/050847 describe the production of recombinant antibodies. By way of further example, mention may also be made of the work by Wei et al. concerning expression of the chimeric antibody chHab18 in the milk of transgenic mice (Transgenic Res. 2011, 20, 321-330). Although transgenic animals produce satisfactory expression levels, extraction and purification of the recombinant protein from the milk remain limiting steps of these expression systems.

Indeed, milk is a complex biological fluid containing about 87% by weight water and 13% by weight solids. It comprises lipids, mainly triglycerides, present in emulsion form; proteins, including casein; sugars, in particular lactose, and secondary components such as vitamins and trace elements. Three phases in milk can generally be distinguished:
whey, comprising carbohydrates, trace elements, serum proteins and water-soluble vitamins, in which are dispersed:
a lipid phase or cream essentially composed of lipids in the form of an emulsion of fat globules of about 2 to 6 μm in diameter, and
a colloidal micellar phase resulting from the association of casein proteins and calcium phosphate salts.

The recombinant protein may, depending on its nature, be present in the whey, be associated with the fat globules and/or be trapped in the casein micelles, which complicates its extraction all the more.

Methods for extracting recombinant antibodies from transgenic milk have been proposed. For example, application WO 97/42835 describes a method for extracting monoclonal antibodies from goats' milk comprising a step of tangential ultrafiltration of raw milk, in the presence of EDTA, followed by chromatography of the permeate on a G protein affinity medium.

Application WO 2004/050847, as for it, mentions that it would be possible to extract the recombinant antibodies present in transgenic milk by implementation of skimming, centrifugation, sedimentation and/or fractionation steps. Nevertheless, application WO 2004/050847 provides no precise exemplary implementation of a purification method. Likewise, application WO 2007/106078 describes a method for extracting a recombinant protein present in transgenic milk comprising a depth-filtration step, but does not propose any concrete application to the purification of recombinant antibodies.

There thus exists, at the present time, a need for new methods for purifying therapeutic recombinant proteins from transgenic milk or from protein solution derived from transgenic milk.

### SUMMARY OF THE INVENTION

The invention relates to a method for purifying a recombinant protein from transgenic milk or from a protein solution obtained from transgenic milk, comprising a clarification step using a poly(diallyldimethylammonium) salt, preferably poly(diallyldimethylammonium chloride) (pDADMAC). In certain embodiments, the recombinant protein is selected from hormones, cytokines, proteins involved in the immune response, antibodies, coagulation factors and coagulation inhibitors. Preferably, the recombinant protein is an antibody.

In certain embodiments of the method according to the invention, the clarification step comprises:
adding the poly(diallyldimethylammonium) salt to said transgenic milk or said solution so as to form a liquid phase comprising the recombinant protein, and a flocculate, and
separating the flocculate and the liquid phase, so as to recover the liquid phase comprising the recombinant protein.

The final poly(diallyldimethylammonium) salt content in the milk or the solution may be in the range of 0.01 g/L to 20.0 g/L. Said milk or said solution may be incubated at a temperature of 20° C. to 60° C., after addition of the poly(diallyldimethylammonium) salt. The separation of the liquid phase and the flocculate may be carried out by a method selected from mechanical pressing and draining.

In certain embodiments, the clarification step is performed on raw transgenic milk having optionally undergone one or more treatments selected from freezing, thawing and pasteurization.

The method according to the invention may further comprise at least one further purification step preferably selected from the group consisting of ultrafiltration, tangential ultrafiltration, microfiltration, diafiltration, reversed-phase chromatography, hydrophobic-interaction chromatography, hydroxyapatite chromatography, cation-exchange chromatography, anion-exchange chromatography, affinity chromatography, multimodal chromatography, size-exclusion chromatography, and combinations thereof.

In an embodiment, the purification method comprises at least one multimodal chromatography step. By way of example, the method may comprise two multimodal chromatography steps.

In another embodiment, the purification method according to the invention comprises a step of elimination and/or inactivation of residual pathogens.

The method according to the invention may comprise one, several, or all of the following features:
the milk solution is raw transgenic milk, and/or
the recombinant protein is a human, humanized or chimeric antibody, and/or
the pDADMA salt is a pDADMAC having an average molecular weight in the range between 100,000 and 500,000 g/mol, and/or
in the precipitation step, the final concentration of pDADMA salt is from 2.0 g/L to 5.0 g/L, and/or
the method comprises at least one multimodal chromatography step performed downstream of the clarification step, and/or
the method comprises at least one purification step selected from microfiltration, ultrafiltration, tangential ultrafiltration, microfiltration, diafiltration, reversed-phase chromatography, hydrophobic interaction chromatography, hydroxyapatite chromatography, cation-exchange chromatography, anion-exchange chromatography, affinity chromatography, and size-exclusion chromatography, and/or
the method comprises a pasteurization step performed upstream of the clarification step, and/or
the step of clarification by addition of pDADMA salt is the only clarification step of the method, and/or
the method does not comprise a skimming step performed upstream of the step of clarification by addition of pDADMA salt.

The purification method according to the invention may, for example, comprise the combination of the following steps:
a. pasteurization of the transgenic milk or of said solution,
b. a step of clarification of the pasteurized transgenic milk or of said pasteurized solution by addition of a pDADMA salt, preferably pDADMAC,
c. implementation of a multimodal chromatography step on the clarified solution obtained in step (c), optionally after adjustment of the pH or the conductivity of said solution,
d. optionally, implementation of one or more further purification steps on the recombinant protein obtained in step (d), preferably selected from the group consisting of ultrafiltration, tangential ultrafiltration, microfiltration, diafiltration, reversed-phase chromatography, hydrophobic interaction chromatography, hydroxyapatite chromatography, cation-exchange chromatography, anion-exchange chromatography, affinity chromatography, size-exclusion chromatography, and combinations thereof, and
e. at least one step of inactivation and/or elimination of residual pathogens, preferably by sterilizing nanofiltration.

The invention also relates to a method for preparing a pharmaceutical composition comprising a recombinant protein, said method comprising:
providing a recombinant protein by performing a purification method according to the invention, as defined above, and
mixing the recombinant protein with one or more pharmaceutically acceptable excipients.

The invention further relates to the use of a poly(diallyldimethylammonium) salt, preferably pDADMAC, as a clarifying agent for purifying a therapeutic protein from transgenic milk or from a protein solution obtained from transgenic milk.

### FIGURES

FIGS. 1A, 1B and 1C show the results of clarification experiments on transgenic milk described in Example 1. The term “Flocculant” refers to pDADMAC.

FIG. 1A shows supernatant clarity for each clarifying agent tested. Clarity corresponds to 1/turbidity, the turbidity of each supernatant having been determined by absorbance at 400 nm. The higher the clarity value, the more effective the clarifying agent.

FIG. 1B shows the recombinant antibody content (mg) in the supernatant obtained for each clarifying agent. The antibody content was determined by nephelometry.

FIG. 1C shows the protein purity in antibody (relative to total protein) in each supernatant; protein purity in antibody was determined from SDS-PAGE gels according to the technique of Laemmli.

FIG. 2 shows the electrophoresis gel obtained by SDS-PAGE (Coomassie blue staining) for supernatants from the various clarification conditions tested and summarized in the table of Example 2. For each compound, the gel on the left corresponds to the “low” concentration tested and the gel on the right corresponds to the high concentration tested.

FIG. 3 shows a diagram describing the steps of a purification method according to the invention. Implementation of this method is exemplified in Example 4.

### DETAILED DESCRIPTION

To the knowledge of the Inventors, poly(diallyldimethylammonium chloride) (pDADMAC) is a flocculant used mainly in the chemicals industry, in particular in the fields of water treatment and papermaking.

The Inventors have shown that, surprisingly, poly(diallyldimethylammonium chloride) (pDADMAC) can be used as a clarifying agent for purifying a therapeutic recombinant protein from transgenic milk. Indeed, pDADMAC makes it possible to precipitate a large proportion of the casein micelles and the fat globules present in milk while retaining the recombinant protein in solution.

In particular, the Inventors have shown that addition of pDADMAC to transgenic milk comprising a recombinant antibody induces the formation of a multiphase system comprising:
a supernatant in the form of a low-turbidity, practically clear solution rich in recombinant antibodies, and
a high-turbidity phase comprising the bulk of the casein micelles and the fat globules of the starting milk, in the form of a flocculate (also called floc or milk sediment).

The recombinant antibody-rich supernatant can be collected by a conventional liquid-solid separation step, for example by a step of mechanical pressing, draining or filtration.

Thus, by a single clarification step, the Inventors obtained a pre-purified recombinant antibody solution, free of virtually all the fat globules and caseins present in the starting milk and having a turbidity lower than 0.1, with a recombinant antibody yield of about 80%.

### General Embodiment of the Purification Method According to the Invention

Thus, a first object according to the invention is the use of a poly(diallyldimethylammonium) salt as a clarifying agent for purifying a recombinant protein from transgenic milk or from a protein solution obtained from transgenic milk.

More precisely, the invention relates to a method for purifying a recombinant protein from transgenic milk or from a protein solution obtained from transgenic milk, comprising a clarification step using a poly(diallyldimethylammonium) salt.

Within the meaning of the invention, by “clarification” is meant the fact of decreasing the turbidity of a solution. In other words, a clarification step decreases the quantity of suspended particles in a solution. In the case of a milk solution, the clarification step removes, at least partially, the casein colloids and/or the fat globules in suspension.

Within the meaning of the invention, by “recombinant protein” is meant a protein whose expression in the transgenic animal results from the introduction of a heterologous gene into its genome. Methods for producing transgenic animals expressing a recombinant protein in their milk are well-known to a person skilled in the art. By way of example, a person skilled in the art may refer to patent EP 0 741 515, to application WO 2004/050847, or to the review article by Houdebine (2009; see above), the teachings of which are incorporated herein by reference. The heterologous gene encoding the recombinant protein is preferably under the control of a promoter directing its expression in mammary epithelial cells. For example, the promoter may be, but is not limited to, a casein promoter, a lactalbumin promoter, a whey acidic protein (WAP) promoter, or a beta-lactoglobulin promoter. The transgenic animal may be selected from a goat, a cow, a rabbit, a sow, a mouse, a ewe or a camel. Preferably, the transgenic animal is a goat, a sow or a rabbit.

The recombinant protein may be of any type. Preferably, it is a recombinant protein intended for use in human or veterinary medicine. It may be a human protein or a variant of a human protein.

By way of example of “recombinant proteins” of interest that one wishes to express in a transgenic animal, mention may be made of hormones, cytokines, proteins involved in the immune response, for example factor H, therapeutic antibodies, antibody fragments such as Fc, Fab or F(ab′)₂, or single-domain fragments (dAb, nanobodies), as well as coagulation factors and inhibitors. Cytokines include, inter alia, interferons, interleukins, chemokines, tumor necrosis factor and growth factors. Hormones include, inter alia, insulin, erythropoietin, steroid hormones and growth hormone. Coagulation factors include factor VII, (FVII), factor VIII (FVIII), factor X (FX), factor IX (Factor IX), factor XI (FXI), factor XII (FXII), factor XIII (FXIII), factor II (prothrombin), antithrombin III (AT III), heparin cofactor II (HCII), protein C (PC), thrombomodulin (TM), protein S (PS), factor V (FV), von Willebrand factor (vWF) and tissue factor pathway inhibitor (TFPI). According to a particular embodiment, the recombinant protein may be a variant of a wild-type protein, i.e., a protein having one or more mutations relative to the wild-type protein. In another embodiment, the recombinant protein may be a chimeric or fusion protein, for example comprising protein domains from different wild-type proteins.

In a preferred embodiment, the recombinant protein to be purified is a recombinant antibody. The term “antibody” must be understood as encompassing any polypeptide comprising a binding domain derived from a variable region of an immunoglobulin, said binding domain being capable of specifically recognizing an epitope, and comprising one or more CDRs (Complementarity-Determining Regions). Preferably, the recombinant antibody according to the invention comprises at least one variable region coupled to a Fc region. By “Fc” or “Fe region” is meant a polypeptide comprising the constant region of an immunoglobulin (except for the first constant domain, CH1). For IgG antibodies, the Fc region comprises CH2 and CH3 domains and potentially the lower part of the hinge region. The recombinant antibody according to the invention may be a chimeric antibody, a humanized antibody, a human antibody, or a fusion protein comprising at least one variable region of an immunoglobulin fused to a polypeptide of interest, for example a polypeptide with therapeutic activity, a polypeptide that enables cell or tissue targeting, or a polypeptide that facilitates subsequent purification of the antibody. Preferably, the recombinant antibody is a human, chimeric or humanized antibody of G isotype (IgG), for example IgG1, IgG2, IgG3 or IgG4; of A isotype (IgA); of M isotype (IgM); of D isotype (IgD); or of E isotype (IgE); optionally comprising one or more mutations in its Fc region. The recombinant antibody may be a blocking or neutralizing antibody. The recombinant antibody according to the invention may be against any therapeutic target of interest. It may be an antibody against a membrane receptor, a cytokine, an interleukin, a hormone, a toxin, an enzyme, an enzyme cofactor, a viral or bacterial protein, a growth factor, a pathogen, a plasma protein, a cellular protein, a protein of the cell nucleus, DNA or RNA. By way of example but not of limitation, it may be an anti-RhD, anti-CD20, anti-TNF-α, anti-CD137, anti-HBs, anti-tetanus toxin, or anti-CMV antibody.

By way of example of references describing the production of recombinant antibodies in transgenic milk, mention may be made of the article by Pollock et al. (Journal of Immunological Methods, 1999, 231:147-157) or of international application WO 2014125374 in the name of the Applicant, which describes the production of an anti-TNF-α antibody in a transgenic animal. A particularly preferred antibody is an anti-TNF-α antibody such as adalimumab. Within the meaning of the invention, by “transgenic milk” is meant milk obtained from a transgenic animal, i.e., an animal that has been genetically modified to produce a recombinant protein of interest in its milk. Within the meaning of the invention, “transgenic milk” includes transgenic milk having optionally undergone freezing/thawing and/or a sterilization or pasteurization step.

The expression “protein solution derived or obtained from said milk or said transgenic milk,” or called hereinafter “solution obtained from said milk,” refers to a solution that contains the recombinant protein to be purified and that was obtained from transgenic milk by implementation of one or more treatment or purification steps, such as fractionation, virus inactivation and/or supplementation steps. The protein solutions derived from said milk include, but are not limited to, milk in which one or more endogenous components have been at least partially removed, for example skim milk; partially clarified milk; but also milk having undergone virus inactivation or sterilization; milk whose density has been adjusted, for example by dilution; diluted milk, for example by addition of buffer solution; condensed milk; milk supplemented with a preservative, an antioxidant or any other agent, for example an enzyme, a virus-inactivating agent; or milk whose pH and/or conductivity constants have been modified, for example, by addition of an acid, a base and/or an electrolyte.

The solution obtained from transgenic milk contains one or more contaminants, in particular endogenous milk proteins and/or milk lipids. By way of example, said solution or said milk may comprise at least 5 g/L, even at least 10 g/L, of endogenous proteins, notably casein contaminants. In certain embodiments, the mass ratio of “recombinant protein” to “total proteins” in the milk solution is lower than 0.8, even lower than 0.7 or 0.6, for example lower than 0.5, even lower than 0.4 and even lower than 0.3. The weight amount of “total proteins” can be determined by total protein assay techniques such as the Biuret method, the bicinchoninic acid (BCA) assay, the Bradford assay, the Coomassie blue assay, determination of organic nitrogen by the Kjeldahl method, UV or IR absorption, preferably by the BCA assay. The weight amount of “recombinant protein” can be determined by specific immunological techniques such as ELISA, EIA, RIA or nephelometry; or specific biological and/or biochemical techniques such as an enzymatic assay, a cellular assay, a specific binding assay by surface plasmon resonance (on a Biacore system, for example). Preferably, the amount by weight of recombinant protein is determined by ELISA.

“Total proteins” includes the recombinant protein to be purified as well as protein contaminants, in particular caseins and whey proteins.

Preferably, the solution obtained from transgenic milk is in the form of a colloidal solution. For the sake of brevity, the expression “milk solution” refers to both “transgenic milk” and “the protein solution obtained from transgenic milk.”

By “skim milk” is meant milk in which lipids, in particular in the form of fat globules, have been at least partially removed, for example by ultrafiltration, depth filtration or centrifugation. Within the meaning of the invention, the expression “partially removed an entity present in a solution” refers to the fact of reducing the amount of this entity by at least 5%, preferably by at least 10%, for example by at least by 20%, 30%, 40%, 50%, 60%, or by at least 70%, even by at least 80% or 90%, relative to the initial amount of this entity in the solution

Within the meaning of the present invention, by “poly(diallyldimethylammonium) salt” (hereinafter pDADMA salt) is meant a polymer of diallyldimethylammonium, i.e., comprising the following structure:

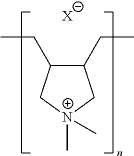

wherein n represents the number of monomer units and X⁻ is a counter-anion. Preferably, n is greater than or equal to 100, preferably less than or equal to 5,000.

pDADMA polymers are generally in the form of a population of polymers.

Preferably, the pDADMA salt has an average degree of polymerization—i.e., an average number of monomer units per polymer chain—in the range of 500 to 4,000, preferably from 1,000 to 3,000.

X⁻ is preferably selected from pharmaceutically acceptable anions. For example, X⁻ may be selected from the following anions: a halide, in particular a chloride or a bromide, a sulfate, a bisulfate, an acetate, a maleate, a bisulfate, a phosphate, a carbonate, or combinations thereof.

Preferably, X⁻ is a halide, in particular Cl⁻.

A particularly preferred poly(diallyldimethylammonium) salt within the meaning of the invention is poly(diallyldimethylammonium chloride), hereinafter called pDADMAC.

A suitable pDADMAC typically has an average molecular weight in the range of 70,000 g/mol to 700,000 g/mol, preferably in the range of 100,000 to 500,000 g/mol, for example 200,000 to 350,000 g/mol.

The clarification step is performed under conditions allowing the flocculation, even the precipitation, at least partially, of one or more components of the milk while retaining, in a soluble state, at least a portion of the recombinant protein, for example the antibody, initially present in the starting solution.

By “flocculation” is meant the fact of inducing agglomeration of particles suspended in the milk solution, such as fat globules and casein micelles, so as to generate larger particles called flocculates or flocs.

By “component of the milk” is meant any endogenous component of the milk, such as casein, whey proteins such as β-lactoglobulin, α-lactalbumin, albumin or lactoferrin; or milk lipids, in particular triglycerides. Preferably, the clarification step removes, at least partially, the casein micelles and the lipids in the form of fat globules initially present in said milk or said solution. Typically, the pDADMA salt may be added to the milk solution either in pure form or in the form of a solution. The addition may optionally be carried out with shaking, at a temperature in the range between 2° C. and 40° C. or at a higher temperature, for example at a temperature of 40° C. to 60° C. Addition of the pDADMA salt may be divided up or spread over time, for example over a period of a few minutes to an hour.

After addition of the pDADMA salt, the milk solution may be incubated at a temperature in the range between 2° C. and 60° C., the time necessary to obtain the flocculate, typically for a few minutes to a few hours, for example for 10 minutes to 24 hours. Advantageously, the addition and incubation temperature is in the range of 20° C. to 50° C., preferably 30° C. to 50° C. A particular preferred temperature is in the range of 35° C. to 50° C. A suitable addition and incubation temperature is 37° C.±1° C. or 45° C.±1° C., for example.

The duration of the incubation step may be in the range of 3 hours to 22 hours, for example 6 hours to 12 hours. The incubation step may be carried out with shaking, or in the absence of any shaking. In certain embodiments, after addition of the pDADMA salt, the medium may be homogenized by shaking for a few minutes to a few hours, even for about 1 to 2 hours, at the desired incubation temperature. Incubation is then continued without shaking, at the desired temperature for the time necessary for satisfactory clarification, typically for 3 hours to 18 hours.

The amount of pDADMA salt to be added in the clarification step depends on the composition of the milk solution. Generally, a final concentration of pDADMA salt in the range of 0.01 g/L to 20.0 g/L is sufficient to obtain satisfactory clarification. Preferably, the concentration of pDADMA salt is in the range of 0.5 g/L to 15.0 g/L, for example 1.0 g/L to 10.0 g/L. A suitable final concentration of pDADMA salt may be in the range of 1.0 g/L to 5.0 g/L, for example in the range of 2 g/L to 5 g/L, even in the range of 3.0 g/L to 4.0 g/L, in particular for pDADMAC. The final concentration of pDADMA salt may be adjusted as a function of protein concentration and milk density. In general, the concentration of pDADMA salt is less than or equal to 4 g/L. The Inventors have shown that the initial pH of the milk solution (i.e., the transgenic milk or the solution obtained from the milk) has no significant effect on the capacity of the pDADMA salt to precipitate casein micelles and fat globules. pDADMA is generally able to flocculate casein micelles and fat globules from milk in a pH range of 5.0 to 11.0; in particular at pH 6.0 to 10.0. The initial pH of the solution may thus be selected as a function of the solubility properties of the recombinant protein to be purified. By way of example, an initial pH in the range of 6.0 to 10.0, typically 6.5 to 9.5, is suitable for purifying a recombinant immunoglobulin from transgenic milk.

In a preferred embodiment, the pDADMA salt is added in the form of an aqueous solution typically having a pDADMA salt concentration of 50 g/L to 450 g/L, for example 100 g/L to 400 g/L. Such solutions, in particular aqueous pDADMAC solutions, are available commercially.

As explained above, addition of the pDADMA salt causes flocculation of casein micelles and lipids from the milk, the recombinant protein remaining mostly in soluble form, in the aqueous phase. The mixture thus obtained may be used directly in a subsequent purification step. Nevertheless, in order to improve the overall yield of the purification method, it is preferable to remove the flocculate formed following addition of the pDADMA salt before implementing subsequent purification steps.

Thus, in a preferred embodiment, the clarification step further comprises a separation step directed at eliminating the flocculate formed.

In other words, the clarification step may comprise:
addition of a poly(diallyldimethylammonium) salt to the milk solution so as to form flocculate, and
removal of the flocculate thus formed, so as to recover the solution comprising the recombinant protein.

The step of flocculate removal may be preceded by a step of incubation of the milk solution as described above. The separation of the flocculate and the aqueous phase comprising the recombinant protein may be carried out by any separation method known to a person skilled in the art. By way of example, a conventional liquid-solid separation technique used in the food processing industry, such as draining or mechanical pressing, may be used. If need be, the separation step may be carried out by centrifugation and/or filtration, for example by tangential filtration, for example tangential microfiltration, depth filtration and combinations thereof. Within the meaning of the invention, by “depth filtration” is meant a filtration method in which the totality of the filter bed is used to trap particles suspended in the fluid. The fluid thus crosses the filter bed in its totality, the particles being trapped on the surface of the filter bed as well as in the filter bed's voids and/or pores. Depth filtration may be performed on cellulose fiber-based, regenerated cellulose-based or polypropylene-based matrices, or combinations thereof. These filter matrices may comprise inorganic compounds such as pearlite, diatomaceous earth, for example kieselguhr, and fumed silica. By way of example, the matrix used for depth filtration may comprise cellulose, propylene, pearlite and kieselguhr. The porosity of the matrix may be in the range of 10 μm to 100 μm, for example 20 m to 60 μm.

As mentioned above, it is also possible to use a tangential filtration step in the clarification step according to the invention. In certain embodiments, this tangential filtration step is performed in combination with, and in particular downstream of, a floc-removal step selected from a pressing step, a draining step and a depth-filtration step. In other embodiments, the tangential filtration step is the only step used to remove the flocculate. All the membranes suitable for tangential filtration, in particular inorganic ceramic membranes, may be used in the method according to the invention. The ceramic membrane may have a porosity threshold of 0.1 to 1.5 μm, for example 0.2 to 1.4 μm, preferably 0.2 μm. Such a ceramic membrane makes it possible to collect a filtrate advantageously free of microorganisms.

The Inventors have shown that, in certain cases, implementation of a sophisticated separation step, such as depth filtration or tangential filtration, was not necessary to remove the flocculate in an optimal manner. The use of a simple separation technique such as draining, pressing or vacuum pumping through a membrane, a frit or a cloth, for example a woven cloth such as nylon cloth, does not diminish the clarity and the purity of the recombinant protein solution collected. Thus, in certain embodiments, the floc-removal step is performed:
by mechanical pressing: mechanical pressing may be carried out, for example, using a bag made of cloth, such as nylon cloth. The nylon cloth may be positioned in a pressure-resistant support, for example made of stainless steel, allowing the clarified liquid to be collected, or
by draining: draining may be also carried out using a cloth, preferably a nylon cloth, positioned in a support, for example made of composite, allowing the clarified liquid to pass freely through said support by simple gravity. The porosity of the cloth used for draining is typically lower than 50 μm, for example lower than 30 μm.

The clarification step according to the invention is generally characterized by a recombinant protein yield of at least 60%, preferably at least 70%.

Typically, implementation of the clarification step decreases the turbidity of the solution by at least a factor of 10, preferably by a factor of 100, indeed by a factor of 1,000. Preferably, as illustrated in the examples, turbidity is determined by spectrophotometry at an absorbance wavelength of 400 nm. Turbidity can also be measured by light scattering, for example in a turbidimeter, an apparatus for normalizing turbidity in NTU (normal turbidity units).

The clarification step generally decreases the turbidity of the solution to a value measured by absorbance at 400 nm lower than 0.8, preferably lower than 0.5, more preferably lower than 0.4, for example lower than 0.3 or lower than 0.2.

The clarification step can also decrease the amount of casein by at least 20%, preferably by at least 30%, for example by at least 40%, 50%, 60%, 70%, 80%, even by at least 90%, relative to the initial amount of casein present in the solution, before implementation of the clarification step.

In the context of the present invention, protein contents, for example recombinant protein or casein contents, can be determined by nephelometry and ELISA.

Alternatively or additionally, the clarification step can decrease the amount of lipids, in particular triglycerides, by at least 20%, preferably by at least 30%, for example by at least 40%, 50%, 60%, 70%, 80%, even by at least 90%, relative to the initial amount of lipids present in the solution, before implementation of the clarification step. The amount of lipids can be measured by colorimetric assay, for example.

The method for purifying a recombinant protein according to the invention may comprise one or more further steps which may be performed upstream or downstream of the step of clarification by addition of pDADMA salt.

By way of example, the method according to the invention may comprise one or more steps selected from the group consisting of:
a freezing/thawing step or a thawing step (for example if the milk solution is provided in frozen form),
a step of removal or inactivation of a pathogen, for example a step of sterilization, pasteurization, and/or virus inactivation,
a skimming step,
a step of adjusting one or more parameters of the milk solution such as pH, conductivity, osmolarity, density or total protein concentration,
   this step or these steps being carried out preferably upstream of the clarification step.

By “skimming step” is meant a step directed at removing, at least partially, the lipids in suspension form potentially present in the starting solution. This skimming step may be carried out by centrifugation. If need be, the skimming step may be carried out by lipid separation, i.e., by allowing the milk to rest sufficiently to obtain two phases. Since the relative density of the cream (milk lipids) is lower than that of water, the lipid layer rises to the surface, making it possible to collect skim milk in the lower phase.

In a particular embodiment, the method according to the invention does not comprise a skimming, settling and/or clarification step performed upstream of the clarification step by addition of pDADMA. In other words, the clarification step according to the invention is carried out on transgenic milk that has not been skimmed, settled or clarified.

Thus, the step of clarification by addition of pDADMA salt may be carried out on raw transgenic milk having optionally undergone one or more preferably physical treatments such as freezing/thawing or sterilization, for example pasteurization. It may also be transgenic milk having undergone virus inactivation, for example by addition of a suitable agent such as a detergent; milk supplemented with a preservative, an antioxidant or any other diluent; or milk whose pH and/or conductivity constants have been modified, for example by addition of an acid, a base and/or an electrolyte.

In a particular embodiment, the step of clarification by addition of pDADMAC is the only clarification step of the method.

By way of example, the method according to the invention may comprise the following steps:
i. optionally, a step of sterilization of the transgenic milk or the solution obtained from said milk,
ii. optionally, a step of adjusting a parameter of the milk solution, in particular its pH, and
iii. a step of clarification by addition of pDADMA salt as described above, said method preferably not comprising a clarification, settling and/or skimming step upstream of step (iii).

In an additional or alternative embodiment, the purification method according to the invention may comprise one or more steps directed at increasing the purity of the recombinant antibody. These steps are preferably carried out downstream of the clarification step. These steps can enable the removal of residual traces of lipids or caseins. Alternatively or additionally, these steps can enable the removal of one or more other contaminants present in the milk solution, such as certain whey proteins (β-lactoglobulin, α-lactalbumin, etc.).

Thus, the method according to the invention may comprise one or more chromatography and/or filtration steps directed at removing or decreasing one or more contaminants. It may be microfiltration, ultrafiltration, tangential ultrafiltration, microfiltration, diafiltration, reversed-phase chromatography, hydrophobic-interaction chromatography, hydroxyapatite chromatography, cation-exchange chromatography, anion-exchange chromatography, affinity chromatography, multimodal chromatography, size-exclusion chromatography, and combinations thereof.

Preferably, the further purification step(s) is/are selected from the group consisting of cation-exchange chromatography, anion-exchange chromatography, affinity chromatography, multimodal chromatography, and the highly salt-tolerant versions thereof.

A particularly preferred technique is multimodal chromatography.

By “multimodal chromatography” (also called “mixed-mode chromatography”) is meant a chromatography method that utilizes several types of interactions between the stationary phase (or resin) and the compounds to be separated. By way of example, multimodal chromatography may be based on implementation of hydrophobic and electrostatic interactions, the stationary phase in this case having ion-exchange properties and properties specific to hydrophobic interaction resins. Other types of interaction may play a part, for example by hydrogen bonding or via thiol functions.

Resins suitable for multimodal chromatography are well-known to a person skilled in the art. Typically, they are resins comprising on their surface a ligand capable of establishing several types of interactions, such as 4-mercapto-ethyl-pyridine and benzyl-methyl-ethanolamine. By way of example, for purifying a recombinant antibody, a commercial resin—such as MEP HyperCel, HEA HyperCel or PPPS HyperCel (Millipore), or Capto MMC or Capto adhere (GE Healthcare)—may be used.

In a preferred embodiment, the method according to the invention comprises at least one multimodal chromatography step. This multimodal chromatography step may be carried out under conditions making it possible to selectively capture the recombinant protein on the surface of the stationary phase, the contaminants (other proteins and/or lipids) present in the solution being not or poorly retained on the stationary phase, under the loading conditions used. The recombinant protein may then be detached and thus recovered from the stationary phase by modifying the properties of the elution solution, for example by modifying the pH. If need be, this multimodal chromatography step may be carried out under so-called “negative” conditions under which the recombinant protein is not retained during loading, whereas the contaminants are. The recombinant protein can thus be recovered directly in the loading effluent.

In the context of purification of a recombinant antibody, it is preferable that the pH of the clarified solution is between 5 and 10, typically a pH of about 6 to 9 for implementation of multimodal chromatography, in particular for resins having 4-mercapto-ethyl-pyridine and benzyl-methyl-ethanolamine as ligand. As illustrated in the examples, the recombinant antibodies present in the clarified solution are strongly adsorbed on resin based on 4-mercapto-ethyl-pyridine and can then be eluted by decreasing the pH of the elution solution to a value lower than 5, for example pH 4. Resin based on benzyl-methyl-ethanolamine, in turn, allows implementation of “negative” multimodal chromatography, the recombinant antibodies present in the clarified solution not being retained on the resin at pH of about 7, unlike the protein contaminants which are strongly adsorbed.

In another additional or alternative embodiment, the method according to the invention may comprise one or more further steps directed at improving the sanitary quality of the final recombinant protein composition, i.e., directed at removing and/or inactivating pathogens potentially present in the milk solution. These pathogens include bacteria, viruses and unconventional pathogens such as prions or endotoxins. The step of inactivation and/or removal of potential pathogens may be implemented according to any technique known to a person skilled in the art. This step of inactivation and/or removal of pathogens may be selected from the group consisting of UV irradiation; gamma irradiation; heat treatment, for example pasteurization; treatment with a chemical agent, for example with a solvent and/or a detergent; sterilizing nanofiltration; and combinations thereof. Sterilizing nanofiltration generally refers to filtration through a filter having a pore size smaller than 80 nm, preferably between 15 and 50 nm. Nanofiltration may be carried out on a single filter or on a series of several filters of decreasing pore size.

In certain embodiments, the method for purifying a recombinant protein according to the invention comprises one or more (2, 3, 4, 5, 6, 7, 8, 9, 10, 11, 12, 13 or 14) of the following features:
the milk solution is transgenic milk,
the recombinant protein is a recombinant antibody,
the pDADMA salt has an average degree of polymerization in the range of 500 to 4,000,
the pDADMA salt is pDADMAC, said pDADMAC preferably having an average molecular weight in the range of 70,000 g/mol to 700,000 g/mol, for example 100,000 to 500,000 g/mol,
the clarification step comprises incubation of the milk solution, after addition of the pDADMA salt,
in the clarification step, addition of the pDADMA salt and/or incubation of the solution is/are carried out at a temperature of 30° C. to 60° C.,
in the precipitation step, the final concentration of pDADMA salt is from 1.0 g/L to 10.0 g/L, preferably from 1.0 g/L to 5.0 g/L, more preferably from 2.0 g/L to 5.0 g/L,
in the clarification step, the flocculate formed is removed by a method selected from mechanical pressing and draining, preferably using a woven nylon cloth,
the method comprises at least one chromatography step, preferably at least one multimodal chromatography step, carried out downstream of the step of clarification by addition of pDADMA salt,
the method comprises at least one purification step selected from microfiltration, ultrafiltration, tangential ultrafiltration, microfiltration, diafiltration, reversed-phase chromatography, hydrophobic-interaction chromatography, hydroxyapatite chromatography, cation-exchange chromatography, anion-exchange chromatography, affinity chromatography and size-exclusion chromatography,
the method comprises a pathogen-removal step performed downstream of the step of clarification by addition of pDADMA salt,
the method optionally comprises a pasteurization step performed upstream of the step of clarification by addition of pDADMA salt,
the step of clarification by addition of pDADMA salt is the only clarification step of the method,
the method does not comprise a skimming step performed upstream of the step of clarification by addition of pDADMA salt.

Needless to say, the method according to the invention may comprise one or more steps in addition to those mentioned above. The method may comprise a step of recovery of the purified recombinant protein or of a purified recombinant protein concentrate.

The method may also comprise steps directed at modifying or adjusting the protein concentration, the conductivity or the pH of the solution before implementation of a purification step such as chromatography. By way of example, the method according to the invention may comprise one or more steps of addition of an acid, a base or a buffer, or dialysis, reverse osmosis, a concentration step, or a dilution step.

The method according to the invention may also comprise one or more steps of conditioning the recombinant protein or the recombinant protein concentrate obtained at the conclusion of the purification steps, in particular a step of formulating the recombinant protein with one or more excipients, preferably one or more stabilizers.

By way of further examples, in particular in the context of purification of a recombinant antibody from transgenic milk, the method according to the invention may comprise a step directed at removing endogenous immunoglobulins secreted in the milk, i.e., immunoglobulins other than the recombinant antibody potentially present in the milk. Removal of the endogenous immunoglobulins may be carried out by affinity chromatography, for example by using an affinity resin capable of selectively retaining the recombinant antibody, the recombinant antibody then being recovered by elution; or an affinity resin capable of selectively retaining endogenous immunoglobulins. By way of example, the affinity matrix may comprise a ligand capable of selectively binding endogenous immunoglobulins; this ligand may be an antibody or an antibody fragment.

### Examples of the Purification Method According to the Invention

According to an additional aspect, the invention relates to a method for purifying a recombinant protein, preferably a recombinant antibody, present in transgenic milk or in a protein solution obtained from said milk, said method comprising any one of the following combinations of steps:

### Combination 1

a. a step of clarification of the milk solution by adding a pDADMA salt, preferably pDADMAC,
b. at least one purification step selected from the group consisting of multimodal chromatography, ultrafiltration, tangential ultrafiltration, microfiltration, diafiltration, reversed-phase chromatography, hydrophobic-interaction chromatography, hydroxyapatite chromatography, cation-exchange chromatography, anion-exchange chromatography, affinity chromatography, size-exclusion chromatography, and combinations thereof, and
c. at least one step of inactivation and/or removal of residual pathogens.

### Combination 2

a. a step of clarification of the milk solution by addition of a pDADMA salt, preferably pDADMAC,
b. at least one multimodal chromatography step,
c. optionally, one or more further purification steps selected from the group consisting of ultrafiltration, tangential ultrafiltration, microfiltration, diafiltration, reversed-phase chromatography, hydrophobic-interaction chromatography, hydroxyapatite chromatography, cation-exchange chromatography, anion-exchange chromatography, affinity chromatography, size-exclusion chromatography, and combinations thereof, and
d. at least one step of inactivation and/or removal of residual pathogens.

### Combination 3

a. pasteurization of the transgenic milk or of said solution,
b. a step of clarification of the pasteurized transgenic milk or of said pasteurized solution by addition of a pDADMA salt, preferably pDADMAC,
c. implementation of a multimodal chromatography step on the clarified solution obtained in step (b), optionally after adjustment of the pH or the salinity of said solution,
d. optionally, implementation of one or more further purification steps on the recombinant protein solution obtained in step (c), preferably selected from the group consisting of ultrafiltration, tangential ultrafiltration, microfiltration, diafiltration, reversed-phase chromatography, hydrophobic-interaction chromatography, hydroxyapatite chromatography, cation-exchange chromatography, anion-exchange chromatography, affinity chromatography, size-exclusion chromatography, and combinations thereof, and
e. at least one step of inactivation and/or removal of residual pathogens, preferably by sterilizing nanofiltration.

### Combination 4

a. provision of transgenic milk, said recombinant protein being a recombinant antibody,
b. pasteurization of the transgenic milk, preferably at a temperature of 45° C. to 65° C.,
c. a step of clarification of the pasteurized transgenic milk by addition of pDADMAC, said step preferably comprising:
   a step of addition of pDADMAC to the transgenic milk so as to attain a final pDADMAC concentration of 1.0 g/L to 5.0 g/L, preferably 2.0 g/L to 5.0 g/L,
   a step of incubation of said transgenic milk after addition of pDADMAC, preferably at a temperature in the range between 30° C. and 60° C., and
   a step of separation of the flocculate formed and the liquid phase, preferably by a technique selected from tangential filtration, depth filtration, pressing and draining, more preferably by pressing or draining, for example through a woven nylon cloth,
d. a multimodal chromatography step performed on the clarified solution obtained in step (c), said multimodal chromatography being implemented preferably on resin having 4-mercapto-ethyl-pyridine or benzyl-methyl-ethanolamine as ligand and a loading pH of 6 to 9,
e. optionally, at least one chromatography step selected from hydrophobic-interaction chromatography, hydroxyapatite chromatography, cation-exchange chromatography, anion-exchange chromatography, size-exclusion chromatography,
f. an ultrafiltration step, preferably on a polyethersulfone membrane,
g. a step of inactivation and/or removal of residual pathogens, preferably by sterilizing nanofiltration, and
h. recovery of the purified recombinant protein or of a purified protein concentrate.

### Combination 5

a. a step of clarification of the milk solution by addition of a pDADMA salt, preferably pDADMAC,
b. a multimodal chromatography step which is implemented under conditions in which the recombinant protein is not absorbed on the matrix at the loading pH and is recovered in the effluent,
c. a second multimodal chromatography step which is implemented under conditions in which the recombinant protein is absorbed on the resin at the loading pH and is then eluted by modification of the pH of the elution solution, wherein step (b) may be performed upstream or downstream of step (c).

### Combination 6

a. provision of transgenic milk or of a protein solution obtained from said milk, where the recombinant protein is a recombinant antibody,
b. a step of clarification of the milk solution by addition of a pDADMA salt, preferably pDADMAC,
c. a first multimodal chromatography step implemented on resin having benzyl-methyl-ethanolamine as ligand, at a pH in the range between 6 and 9, wherein the unabsorbed recombinant antibody is collected in the effluent,
d. a second multimodal chromatography step performed on resin having 4-mercapto-ethyl-pyridine as ligand, with a loading pH in the range between 6 and 9, wherein the recombinant antibody is absorbed on the resin and is then eluted by acidification of the eluent to pH below 5, preferably to pH 4.

### Combination 7

a. provision of transgenic milk or of a protein solution obtained from said milk, where the recombinant protein is a recombinant antibody,
b. a step of clarification of the milk solution by addition of a pDADMA salt, preferably pDADMAC,
c. a first multimodal chromatography step performed on resin having 4-mercapto-ethyl-pyridine as ligand, with a loading pH in the range between 6 and 9, wherein the recombinant antibody is absorbed on the resin and is then eluted by acidification of the eluent to pH below 5, preferably to pH 4,
d. a second multimodal chromatography step implemented on resin having benzyl-methyl-ethanolamine as ligand, at a pH in the range between 6 and 9, wherein the unabsorbed recombinant antibody is collected in the effluent.

It goes without saying that the method according to the invention may comprise one or more steps in addition to those listed in each combination. It goes without saying that the various steps of the method, in particular the clarification step, may be implemented according to any one of the embodiments described above, unless otherwise stated in the combination concerned.

### Additional Objects According to the Invention

According to an additional aspect, the invention also has as an object a recombinant protein or a recombinant protein concentrate obtainable by the purification method as described above. Said recombinant protein or said concentrate is preferably suitable for use in human or veterinary medicine.

An additional object according to the invention is a pharmaceutical composition comprising a purified recombinant protein according to the method described above in combination with one or more excipients. Said pharmaceutical composition may be in liquid or solid form, for example lyophilized.

The invention also has as an object a method for preparing a pharmaceutical composition comprising a recombinant protein, comprising:
i. Providing a purified recombinant protein by the method according to the invention, and
ii. Mixing the recombinant protein with one or more pharmaceutically acceptable excipients.

In certain embodiments, the method for preparing a pharmaceutical composition comprises:
i. Performing the purification method according to the invention, so as to obtain a purified recombinant protein, and
ii. Mixing the purified recombinant protein with one or more pharmaceutically acceptable excipients.

A person skilled in the art will be able to choose the excipient(s) to be combined with the recombinant protein according to the pharmaceutical form and route of administration desired. To this end, a person skilled in the art will be able to refer to the following reference works: Pharmaceutical Formulation Development of Peptides and Proteins (S. Frokjaer and L. Hovgaard, Eds., Taylor & Francis, 2000); Remington: The Science and Practice of Pharmacy (Lippincott Williams & Wilkins; 21^{st} edition, 2005); and Handbook of Pharmaceutical Excipients, American Pharmaceutical Association (Pharmaceutical Press; 6^{th} revised edition, 2009).

The excipient(s) present in the compositions according to the invention may be selected from diluents, cryoprotectants and/or lyoprotectants, stabilizers, antioxidants, pH regulators, buffers, surfactants, detergents, etc. Exemplary excipients include sugars (sucrose, trehalose, glucose, lactose, maltose etc.), polyols (mannitol, sorbitol), amino acids (glycine, arginine, histidine, alanine, methionine), polysorbates (Tween 20 or Tween 80, for example), poloxamers, polyethylene glycol, monothioglycerol, glutathione, citric acid, ascorbic acid, sodium metabisulfite and sodium sulfite, salts of carbonate, phosphate, citrate, acetate, borate, etc.

The pharmaceutical composition according to the invention is preferably intended for parenteral administration, for example via the intravenous, subcutaneous, intradermal or intramuscular route. A particularly preferred route of administration is the intravenous route.

The pharmaceutical composition and the pharmaceutical adjuvant according to the invention may be in any form, for example in the form of a powder, a suspension, a solution, an emulsion, or a lyophilizate, preferably intended for administration to the patient by means of injection.

Finally, the Applicant has also shown that the step of clarification by addition of a pDADMA salt such as pDADMAC had a virus removal and/or inactivation effect, in particular with respect to non-enveloped viruses such as PPV.

Within the meaning of the invention, a virus removal or inactivation effect is deemed to exist when virus reduction by a factor greater than 1 log₁₀ is observed.

Thus, an additional object according to the invention is the use of a pDADMA salt, in particular pDADMAC, for virus inactivation or removal. Preferably, the pDADMA salt is used for virus removal and/or inactivation in a biological solution comprising a protein of interest, for example a cell medium, a cell lysate, a fermentation medium, a biological fluid, and the products derived from said solutions.

In certain embodiments, the pDADMA salt is used as a virus-removing and/or virus-inactivating agent in the purification of a protein from a biological fluid or of a product derived from a biological fluid. The biological fluid may be of any type. For example, it may be blood, blood plasma, including human blood plasma, or milk, for example transgenic milk.

In certain embodiments, the pDADMA salt is used as a virus-removing and/or virus-inactivating agent in a method for purifying a recombinant protein from transgenic milk or a product derived from transgenic milk.

In an additional or alternative embodiment, the pDADMA salt is used as both a virus-removing and/or virus-inactivating agent and a clarifying agent, preferably for purifying a protein of interest from transgenic milk or a product derived from said transgenic milk.

The present invention also has as an object a method for virus removal and/or inactivation in a biological solution comprising a protein of interest, said method comprising a step of incubation of said solution with a pDADMA salt, preferably pDADMAC. This incubation step may be carried out under the conditions described above for the step of clarification using a pDADMA salt according to the invention. Typically, the final concentration of pDADMA salt is lower than 20 g/L, preferably lower than 5 g/L, for example 1 g/L to 4 g/L. The incubation may be carried out at a temperature in the range of 30° C. to 60° C. The incubation time may vary from few minutes to a few hours, typically from 1 h to 20 h.

The purpose of the following examples is to illustrate the invention more fully without limiting its scope.

### EXAMPLES

### Example 1: Evaluation of Several Chemical Agents for Clarifying Transgenic Milk

Transgenic Milk

Transgenic milk is obtained from transgenic goats. The therapeutic recombinant protein secreted in the milk is an anti-TNF-α antibody.

The milk was collected and frozen, for storage with a view to purification.

| Characterization of the milk | Assay | Quantification |
|---|---|---|
| Total protein | BCA | 23.4 g/L |
| Immunoglobulins | ELISA | 2.9 g/L |
| Goats' milk protein (GMP) | ELISA | 26.6 g/L |
| Lactose | Enzymatic | 26.0 g/L |
| Protein composition | HDMse/SDS-PAGE | 46-65% caseins |
| and quantification | | 18-21% Ig |
| | | 9-11% β-lactoglobulin |
| | | 3-5% α-lactalbumin |
| | | 1% lactotransferrin |
| Glass transition temperature | Calorimetry | −25/−50° C. |
| (T_{g}) | | |

Evaluation of pDADMAC and Comparative Tests

pDADMAC was evaluated as a flocculant agent. The stock pDADMAC solution is a commercial solution provided by Merck-Millipore. pDADMAC has an average molecular weight of 100,000 to 500,000 g/mol. For purposes of comparison, putative clarifying agents described in the literature for protein purification were tested under the conditions indicated below.

| | | Final | | |
|---|---|---|---|---|
| | | pH | Final | |
| | | of the | concentration | Commercial |
| | Clarifying agents | milk | in the milk | product |
| Other | Sodium citrate | 7.5 | 200 mM | powder |
| clarifying | Calcium chloride | 5.5 | 200 mM | powder |
| agents | Sodium EDTA | 4.5 | 100 mM | powder |
| (comparative) | Acidic EDTA | 3.5 | 100 mM | powder |
| Invention | pDADMAC | 6.5 | 0.1% (w/v), | 10% (w/v) |
| | | | or about | aqueous |
| | | | 1 g/L. | solution |

Procedure

Each clarifying agent was tested as follows:

Transgenic milk (5 g) was thawed and brought to the desired incubation temperature (4° C., 25° C. or 37° C.). The pH of the milk was adjusted to pH 9.5. For each agent tested, a suitable amount of the aqueous stock solution was added so as to obtain the desired concentration (see table below). After addition of the clarifying agent, the milk was incubated for 20 h, at the desired incubation temperature. At the end of incubation, supernatant samples were taken for analyses. Supernatant turbidity was determined by absorbance at 400 nm. Supernatant clarity is inversely proportional to supernatant turbidity (ratio=1/turbidity).

Recombinant antibody content (mg/L) was determined by nephelometry (anti-total Ig). Protein purity in recombinant antibody was determined by relative measurement of the relative density of immunoglobulin bands after electrophoretic migration in SDS-PAGE according to the technique of Laemmli.

Results

An incubation temperature of 37° C. showed the best results, irrespective of the clarifying agent considered.

Addition of pDADMAC caused very distinct phase separation: the formation of white flocculate, which sediments, and clear supernatant are observed. A similar result was observed for acidic EDTA. In contrast, agents such as CaCl₂, sodium citrate or disodium EDTA did not produce a clear phase.

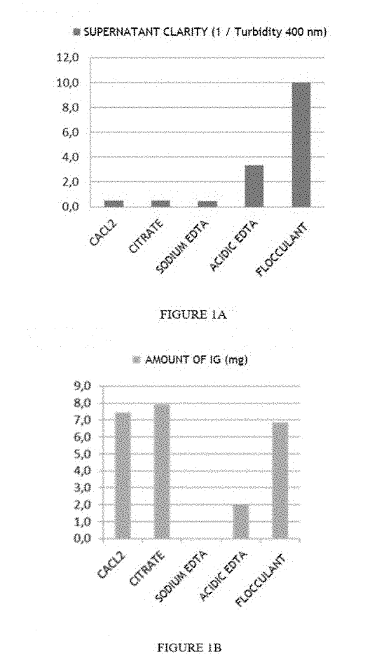

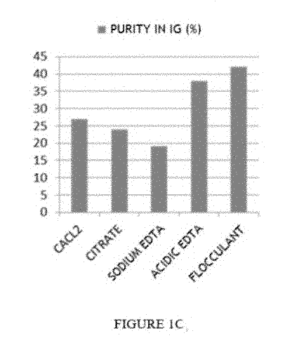

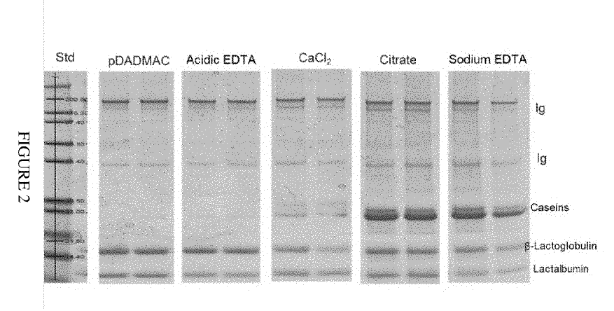

Analyses of the supernatant, for each agent tested, are illustrated in FIGS. 1A-1C for an incubation temperature of 37° C. These analyses confirm that pDADMAC is the most effective agent in terms of clarification (FIG. 1A) and of final purity in recombinant antibody (FIG. 1C).

As illustrated in FIG. 1B, pDADMAC also enables recovery, in the supernatant, of a large proportion of the recombinant antibodies initially present in the transgenic milk.

### Example 2: Optimization of the Clarification Step

The following conditions were tested:

| | Reference final | Initial pH of the milk for |
|---|---|---|
| Solutions | concentrations tested | the clarification step |
| CaCl₂ | 200 mM | 5.5 |
| | 500 mM | |
| Citrate | 215 mM | 7.7 |
| | 500 mM | |
| Sodium EDTA | 100 mM | 4.7 |
| | 300 mM | |
| Acidic EDTA | 100 mM | 3.5 |
| | 300 mM | 4.5 |
| pDADMAC | 0.1% (w/v) (about 1 g/L) | 6.5 |
| (invention) | 0.3% (w/v) (about 3 g/L) | |

The procedure is similar to that used in Example 1. Each clarifying agent was tested as follows: Transgenic milk (100 g) was thawed and adjusted to the incubation temperature, namely 37° C. The thawed milk was subjected to a step of pasteurization at 60° C. for 6 hours in order to neutralize bacteria potentially present in the milk and to avoid bacterial development during incubation with the clarifying agent. The pH of the milk was adjusted to the desired value. For pDADMAC, the initial pH of the milk corresponded to the desired pH, and thus pH adjustment was not needed. For each agent tested, a suitable amount of the corresponding aqueous stock solution was added to the pasteurized transgenic milk. After addition of the clarifying agent and homogenization, the milk was incubated for 20 h without shaking, at 37° C. At the end of incubation, supernatant samples were taken for analyses.

Supernatant turbidity, recombinant antibody content, and protein purity in recombinant antibody were determined as described in Example 1.

Results

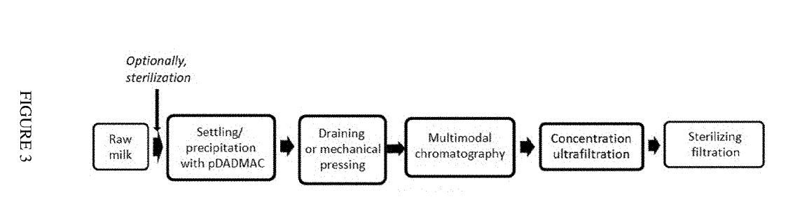

Once again, pDADMAC proved to be the most effective clarifying agent, even in the absence of any adjustment of the initial pH of the solution. Indeed, pDADMAC at a final concentration of 0.1% (w/v) produced a supernatant having very high purity in recombinant antibody and very high clarity. The use of a higher pDADMAC concentration, namely 0.3% (w/v), or 3 g/L, gave equally satisfactory results in terms of antibody purity and content in the supernatant. FIG. 2 shows the electrophoresis gel obtained by SDS-PAGE (Coomassie blue staining) for the supernatants obtained under the various conditions tested. For the supernatants obtained with pDADMAC, the electrophoresis gels show virtually no evidence of caseins, which attests to the removal of caseins in the flocculate. In contrast, the protein bands corresponding to immunoglobulins are quite visible. Such a profile is not observed for the gels of supernatants obtained with CaCl₂, sodium citrate or sodium EDTA, in which casein detection remains high. It should be noted, however, that EDTA in acid form allowed satisfactory removal of caseins. Nevertheless, the amount of immunoglobulin recovered in the supernatant is lower than that obtained with pDADMAC.

### Example 3: Evaluation of the Methods for Collecting Supernatant

Addition of pDADMAC makes it possible to efficiently precipitate casein micelles and fat globules while generating a practically clear supernatant, enriched in recombinant antibody. The supernatant has a clarity such that it is not necessary to subject it to a complex depth filtration step. Several liquid-solid separation techniques were tested. The Inventors have shown that the “pressing liquid”—i.e., the liquid phase obtained by pressing the floc—has a SDS-PAGE profile equivalent to that of the clear supernatant, in particular in terms of protein purity in immunoglobulin.

Such a result led the Inventors to test two simple solid-liquid separation techniques, namely:
Mechanical pressing using a nylon cloth and a porous support with collection by vacuum pumping;
Draining through a nylon cloth having a pore size of 25 μm, by simple gravity.

The filtrates thus obtained have a total protein content of 13.3 g/L, with the following composition determined by SDS-PAGE: 45% immunoglobulins, 0% caseins, and 55% other milk proteins.

The total yield in immunoglobulins of the clarification step (addition of pDADMAC followed by flocculate removal by draining or pressing) is between 75% and 90%, depending on the experiment performed.

As shown in Example 4, the filtrates obtained in the clarification step have a recombinant antibody concentration, a conductivity (lower than 20 mS/cm), and a pH (about 6.5) that allow direct implementation of a multimodal chromatography step, without preliminary adjustment of their composition.

### Example 4: Example of Implementation of a Method According to the Invention

FIG. 3 shows an exemplary method according to the invention.

Procedure

Thawed raw transgenic milk (100 g) was subjected to a step of pasteurization by heating at 60° C. for 10 hours. The milk was then returned to a temperature of 37° C. Aqueous pDADMAC solution (10%, w/v) was added to the pasteurized milk so as to reach a final content of 0.3% (w/v). The transgenic milk was then incubated at 37° C. for 20 hours. No adjustment of the pH of the transgenic milk was performed before addition of pDADMAC. The recombinant antibody-rich supernatant was separated by gravity from the flocculate formed following addition of pDADMAC. The supernatant thus collected was then subjected to a multimodal chromatography step, without preliminary adjustment of its pH, conductivity, or protein concentration. Two types of multimodal chromatography were tested, namely multimodal chromatography by adsorption of Ig on MEP HyperCel resin and multimodal chromatography by adsorption of non-Ig proteins on Capto adhere resin. The recombinant antibody solution obtained in the multimodal chromatography step was purified x times. The antibody solution can then be concentrated by ultrafiltration on a polyethersulfone membrane.

Multimodal Chromatography on MEP Hypercel Resin (Ligand: 4-Mercapto-Ethyl-Pyridine)

Operating conditions tested:
Adsorption: at pH 6.5
Washing: at a conductivity lower than 5 mS/cm
Elution: at pH 4.0

At pH 6.5, the recombinant antibody is strongly absorbed on the resin, whereas the contaminants are not retained. The recombinant antibody is recovered by elution using citric acid solution at pH 4.0.

Multimodal Chromatography on Capto Adhere Resin (Ligand: Benzyl-Methyl-Ethanolamine)

Operating conditions tested: Adsorption: at pH 7.0

At pH 7.0, the recombinant antibody is weakly retained on the resin, whereas the contaminants are. The recombinant antibody is recovered in the non-retained loading eluent fraction.

Multimodal chromatography thus functions in the negative for the recombinant antibody.

Results

The results obtained by the combination of a step of clarification with pDADMAC followed by multimodal chromatography are satisfactory. Both multimodal chromatography techniques tested provide good recombinant antibody yields (about 70-80%).

MEP HyperCel resin allows excellent adsorption of recombinant antibodies (100% in this loading example), leading to a final yield of about 70%. Depending on the load applied, a contaminant protein, casein, can be detected by SDS-PAGE in small amounts in the antibody solution purified by multimodal chromatography on MEP HyperCel resin.

Capto adhere resin allows implementation of negative chromatography, in which the contaminants in the milk are strongly retained on the resin during loading whereas the recombinant antibody is eluted directly at pH 7. Depending on the load applied, residual amounts of two protein contaminants can be detected by SDS-PAGE in the antibody solution purified by multimodal chromatography on Capto adhere resin. The combination of a step of clarification with pDADMAC followed by multimodal chromatography on resin with benzyl-methyl-ethanolamine as ligand makes it possible to completely remove caseins, β-lactoglobulin and fat globules from the milk while having an overall recombinant antibody yield of the two steps of about 60%.

### Example 5: Robustness of the Step of Clarification with pDADMAC as a Function of the Protein Composition of the Starting Transgenic Milk

The clarification method according to the invention was implemented on two transgenic milks having different protein compositions. They are transgenic goats' milks expressing a human antibody. An anti-TNF-α antibody was expressed in milk no. 1. An anti-HER2/neu receptor antibody was expressed in milk no. 2.

These milks have the following features:

| Characterization of the milk | Assay | Milk no. 1 | Milk no. 2 |
|---|---|---|---|
| Total protein | OD 280 nm | 30 g/L | 80 g/L |
| Casein | SDS-PAGE | 18 g/L | 20 g/L |
| Immunoglobulins | Nephelometry | 5 g/L | 52 g/L |

For each milk, a step of clarification using pDADMAC was performed according to a protocol similar to that described in Example 2.

Transgenic milk (100 g) was thawed and subjected to a step of pasteurization at 60° C. for 6 hours in order to neutralize bacteria potentially present in the milk and to avoid bacterial development during incubation with the clarifying agent.

pDADMAC was added so as to obtain a final concentration of about 2 g/L or 4 g/L in solution. After addition of pDADMAC and shaking for 1.5 h at 45° C., the milk was incubated at 45° C. for 6 h to 12 h without shaking. At the end of incubation, supernatant samples were taken for analyses.

The conditions of the procedure are summarized in the following table:

| Clarification | | |
|---|---|---|
| conditions | Milk no. 1 | Milk no. 2 |
| Incubation | +45° C. | +45° C. |
| temperature | | |
| Incubation time | Between 6 and 12 h | Between 6 and 12 h |
| Stirring | 1.5 h at +45° C. | 1.5 h at +45° C. |
| Final pDADMAC | 0.2% or 0.4% (w/v), or | 0.2% or 0.4% (w/v), or |
| concentration | about 2 g/L or 4 g/L | about 2 g/L or 4 g/L |

The following table presents the composition of the raw milks and of the supernatants obtained at the conclusion of the clarification step.

| | Milk no. 1 | Milk no. 2 |
|---|---|---|
| Ig in the supernatant (SDS-PAGE) | 15% | 65% |
| Ig in the clarified milk (SDS-PAGE) | 40% | 80% |
| Caseins in the raw milk (SDS-PAGE) | 60% | 25% |
| Caseins in the supernatant (SDS-PAGE) | 2% | 2% |

The step of clarification with pDADMAC enables significant enrichment in immunoglobulin while effectively removing caseins, and this independently of the initial casein and recombinant immunoglobulin contents of the raw starting milk. The recombinant antibody yield after flocculate removal is at least 50% when the supernatant is collected by gravity (by draining) and at least 70% when rinsing of the flocculate is carried out at the conclusion of draining with a volume of suitable solution.

### Example 6: Virus Inactivation/Removal Effect of the pDADMAC Treatment Step

### 1. Protocol

A volume of transgenic milk (4 g/L) was thawed. The temperature of the thawed product was in the range between 21° C. and 22° C. The thawed milk was then overloaded with 5% (v/v) model virus (PPV). A sample was taken to quantify the amount of virus at this stage. A 3% (v/v) equivalent volume of 10% pDADMAC solution was then added to the volume of milk overloaded with the model virus (a final pDADMAC concentration of about 3 g/L). A sample was taken to quantify the amount of virus at this stage. The solution was then mixed for 1 hour at 45° C. and then incubated for 20 hours at 45° C. A sample was taken to quantify the amount of virus at this stage.

### 2. Results

PPV is a virus representative of small non-enveloped viruses. It is a DNA genome virus, 18-24 nm in size, which is highly resistant to physicochemical treatments. Virus reduction is by a factor of 2.61±0.34 log₁₀ after incubation of the transgenic milk with pDADMAC. This result is chiefly the action of the pDADMAC treatment on this non-enveloped virus.

1.-14. (canceled)

15. A method for purifying a recombinant protein from transgenic milk or from a protein solution obtained from transgenic milk, comprising clarifying the transgenic milk or the protein solution using a poly(diallyldimethylammonium) salt.

16. The method of claim 15, wherein the poly(diallyldimethylammonium) salt is poly(diallyldimethylammonium chloride) (pDADMAC).

17. The method of claim 15, wherein the recombinant protein is selected from the group consisting of hormones, cytokines, proteins involved in the immune response, antibodies, coagulation factors and coagulation inhibitors.

18. The purification method of claim 15, said clarification step comprising:
(i) adding the poly(diallyldimethylammonium) salt to said transgenic milk or to said protein solution to form a liquid phase comprising the recombinant protein and a flocculate, and
(ii) separating the flocculate and the liquid phase, to recover the liquid phase comprising the recombinant protein.

19. The purification method of claim 18, wherein the final poly(diallyldimethylammonium) salt content in the milk or the solution in step (i) is in the range of 0.01 g/L to 20.0 g/L.

20. The purification method of claim 18, wherein, in step (i), said milk or said protein solution is incubated at a temperature of 20° C. to 60° C., after addition of the poly(diallyldimethylammonium) salt.

21. The purification method of claim 18, wherein separation of the liquid phase and a flocculate is carried out by mechanical pressing or draining.

22. The purification method of claim 15, wherein the clarification step is performed on raw transgenic milk having optionally undergone one or more treatments selected from the group consisting of freezing, thawing and pasteurization.

23. The purification method of claim 15, said method comprising at least one further purification step selected from the group consisting of ultrafiltration, tangential ultrafiltration, microfiltration, diafiltration, reversed-phase chromatography, hydrophobic-interaction chromatography, hydroxyapapite chromatography, cation-exchange chromatography, anion-exchange chromatography, affinity chromatography, multimodal chromatography, size-exclusion chromatography, and combinations thereof.

24. The purification method of claim 15, further comprising at least one multimodal chromatography step.

25. The purification method of claim 15, further comprising a step selected from removal of pathogens, inactivation of residual pathogens and combination thereof.

26. The purification method of claim 15, wherein:
the milk solution is raw transgenic milk,
the recombinant protein is a human, humanized or chimeric antibody,
the pDADMA salt is a pDADMAC having an average molecular weight in the range between 100,000 and 500,000 g/mol,
in the precipitation step, the final concentration of pDADMA salt is from 2.0 g/L to 5.0 g/L,
the method comprises at least one multimodal chromatography step performed downstream of the clarification step,
the method comprises at least one purification step selected from the group consisting of microfiltration, ultrafiltration, tangential ultrafiltration, diafiltration, reversed-phase chromatography, hydrophobic-interaction chromatography, hydroxyapatite chromatography, cation-exchange chromatography, anion-exchange chromatography, affinity chromatography, and size-exclusion chromatography,
the method comprises a pasteurization step performed upstream of the clarification step, the step of clarification by addition of pDADMA salt is the only clarification step of the method, or
the method does not comprise a skimming step performed upstream of the step of clarification by addition of pDADMA salt.

27. The purification method of claim 15, further comprising the steps of:
a. pasteurization of said transgenic milk or said solution,
b. a step of clarification of said pasteurized transgenic milk or of said pasteurized solution by addition of a pDADMA salt,
c. a multimodal chromatography on the clarified solution obtained in step (b), optionally after adjustment of the pH or the conductivity of said solution,
d. optionally, one or more further purification steps on the recombinant protein obtained in step (c), selected from the group consisting of ultrafiltration, tangential ultrafiltration, microfiltration, diafiltration, reversed-phase chromatography, hydrophobic-interaction chromatography, hydroxyapatite chromatography, cation-exchange chromatography, anion-exchange chromatography, affinity chromatography, size-exclusion chromatography, and combinations thereof, and
e. at least one step of inactivation or removal of residual pathogens.

28. A method for preparing a pharmaceutical composition comprising a recombinant protein, said method comprising:
providing a recombinant protein by carrying out a purification method of claim 15, and
mixing the recombinant protein with one or more pharmaceutically acceptable excipients.
